(19) United States
(12) Patent Application Publication
(10) Pub. No.: US 2024/0216249 A1
(43) Pub. Date: Jul. 4, 2024

(54) COLLAGEN ACTIVATOR, AND PREPARATION METHOD AND APPLICATION THEREOF

(71) Applicant: Hangzhou NUPTEC Rising Bioproducts Incorporation, Ltd., Hangzhou (CN)

(72) Inventors: Yongchang Qian, Hangzhou (CN); Shiyu Yang, Hangzhou (CN); Qianru Qin, Hangzhou (CN); Tongtong Li, Hangzhou (CN)

(21) Appl. No.: 18/465,179

(22) Filed: Sep. 12, 2023

(30) Foreign Application Priority Data
Dec. 31, 2022 (CN) 2022117401579

(51) Int. Cl. A61K 8/63; A61Q 19/08

(52) U.S. Cl. CPC A61K 8/63; A61Q 19/08

(57) ABSTRACT

A collagen activator, and a preparation method and an application thereof are provided. The collagen activator consists of the following raw materials by mass percentage: 15%-20% of butylene glycol, 1%-4% of 1, 2-hexanediol, 0.5%-2% of dipotassium glycyrrhizinate, 0.1%-2% of sodium acetylated hyaluronate, 0.01%-0.1% of semen coiois extract, 0.01%-0.1% of Bletilla striata extract, 0.001%-0.01% of astragalus extract, and the balance of water. The collagen activator not only can promote an increase of endogenous collagen in skin, but also can promote cell proliferation and cell migration, thereby achieving effects of anti-wrinkle and skin repair. Furthermore, the collagen activator is few in types of the raw materials, is relatively low in cost and relatively high in safety, is used in a smearing mode for convenience, and is suitable for popularization and use.

### TECHNICAL FIELD

The disclosure relates to the technical field of skincare products, particularly to a collagen activator, and a preparation method and an application thereof.

### BACKGROUND

Due to increasing pressures of modern social life and work, people's skin maintains a long-term state of excitement, which means that wrinkled muscles are tense; and therefore, the people's skin appear some faint lines or wrinkles. In addition, under the pressures, people stay up at night frequently, resulting in various skin problems. Under double-sided clamping of the pressures and the skin problems, the people's skin continues aging, losing elasticity and becoming dark and dull. Therefore, skincare becomes a growing concern for the people.

In daily skincare, consumers pay more attention to various active ingredients in the skincare products and choose products that are suitable and safe for their skin types. At present, efficacies of common raw materials in the market are not completely verified, which generates a certain influence on a subsequent application. Alpha hydroxy acids or salicylic acid can regenerate the skin, resulting from peeling off epidermis and dermis shallow layer, thereby improving the dullness and the darkness of the skin and solving a problem of coarse pores. However, acid substances have certain irritation, and frequently or excessively using the acid substances may cause skin sensitivity or even cause skin peeling, redness and swelling, etc. Medical electronic beauty, which utilizes radiation energy and thermal effects of electromagnetic waves, can tighten the skin and increase the skin elasticity. However, side effects of optoelectronic beauty may lead to swelling, allergies, and skin damage. Furthermore, the price of optoelectronic beauty is relatively expensive. Injecting beauty, by locally injecting hyaluronic acid or botulinum toxin to improve skin wrinkles and to fill skin grooves, can make the skin smooth and tight. However, the market for injection beauty is relatively unregulated, and if certain parts of the body are injected inappropriately, it may lead to localized unevenness. Furthermore, injection errors can cause necrosis of a vascular innervation area, necrosis of the skin, and blindness of eyes.

Moreover, people mostly supplement the collagen by means of exogenous sources through oral administration, smearing, or injection. The exogenous source supplement is feasible, but results in a low absorption rate. In addition, whether the exogenous sources can be converted into the collagen is unknown. Therefore, a core of an anti-aging product is to increase endogenous collagen, thereby realizing a significant conversion. Therefore, there is an urgent need to develop a raw material for the skincare product that is capable of promoting the increase of endogenous collagen.

### SUMMARY

In order to solve the above-mentioned problems in the related art, the disclosure provides a collagen activator, and a preparation method and an application thereof, thereby providing a compound safe and effective raw material formula that is capable of promoting increases of endogenous collagen I, endogenous collagen III, and endogenous collagen XVII in the skin, further achieving a purpose of anti-aging.

In order to achieve the above objective, the disclosure provides the following technical solutions.

One of the technical solutions of the disclosure is a collagen activator, consisting of the following raw materials by mass percentage: 15%-20% of butylene glycol (C₄H₁₀O₂), 1%-4% of 1, 2-hexanediol (C₆H₁₄O₂), 0.5%-2% of dipotassium glycyrrhizinate (C₄₂H₆₃KO₁₆), 0.1%-2% of sodium acetylated hyaluronate, 0.01%-0.1% of semen coiois extract, 0.01%-0.1% of Bletilla striata extract, 0.001%-0.01% of astragalus extract, and the balance of water.

Another one of the technical solutions of the disclosure is a preparation method of the collagen activator, including the following steps: weighing the raw materials and mixing the raw materials to obtain the collagen activator.

Still another one of the technical solutions of the disclosure is an application of the collagen activator in a skincare product.

In an embodiment, the skincare product is an anti-aging skincare product.

Still another one of the technical solutions of the disclosure is an anti-aging skincare product, including the collagen activator described above.

In an embodiment, a content of the collagen activator in the anti-aging skincare product is in a range of 3 wt % to 15 wt %.

Sodium acetylated hyaluronate (AcHA) is obtained by an acetylation reaction of a natural moisturizing factor, i.e., sodium hyaluronate (C₁₄H₂₂NNaO₁₁, abbreviated as HA). An introduction of an acetyl group increases a lipophilic property to the HA, enhances an affinity and absorption of the HA to the skin, and improves a moisturizing performance of the HA. The AcHA has effects of efficient moisturizing, repairing a skin barrier, and increasing skin elasticity. And the AcHA makes the skin feel refreshing and non-sticky. Compared with traditional moisturizers, the AcHA has a better moisturizing effect and has no drawbacks such as greasy sensation and clogging of skin pores. When sodium hyaluronate is added to cosmetics and applied to the skin, a layer of viscoelastic transparent hydration film is formed. The film has a good water retention effect, just like a naturally occurring sodium hyaluronate in an interstitial substance, which can increase the moisture of the skin's stratum corneum. Furthermore, the moisture of the skin's stratum corneum is closely related to the maintenance of skin health and defense against external irritation. In addition, a part of the sodium hyaluronate can permeate into the dermis to regulate skin metabolism and to increase skin moisture, smoothness, delicacy, and softness, thereby playing a role in anti-wrinkle beauty and health care.

It is proved in cell experiments that a content of the dipotassium glycyrrhizinate being 0.001%-0.1% has a promoting effect on an expression of the collagen I and an optimal content is 0.01%; a content of the dipotassium glycyrrhizinate being 0.001%-0.25% has a promoting effect on an expression of the collagen III and an optimal content is 0.1%; and a content of the dipotassium glycyrrhizinate being 0.001%-0.01% has a promoting effect on an expression of the collagen XVII and an optimal content is 0.01%. Furthermore, a content of the sodium acetylated hyaluronate being 0.001%-0.05% has a promoting effect on the expression of the collagen I and an optimal content is 0.005%; a content of the sodium acetylated hyaluronate being 0.001%-0.05% has a promoting effect on the expression of the collagen III and an optimal content is 0.005%; and a content of the sodium acetylated hyaluronate being 0.001%-0.05% has a promoting effect on the expression of the collagen XVII and an optimal content is 0.025%.

By adopting an in-vitro experiment combined with a human body experiment, studies are conducted on effects of whitening, anti-aging, anti-allergic and soothing of a compound extract made from three Chinese herbal medicines (i.e., the semen coiois extract, the Bletilla striata extract, and the astragalus extract), and its irritation is evaluated. Results show that the compound extract has no irritation, and has strong ability to inhibit an activity of tyrosinase, to remove 1, 1-diphenyl-2-picrylhydrazyl (C₁₈H₁₂N₅O₆, abbreviated as DPPH)/hydroxyl/superoxide anion free radicals, and to inhibit an activity of hyaluronidase. The above results show that the compound extract is high in use safety, and has perfect effects on whitening, anti-aging, anti-allergy, and soothing.

Cell experiments are performed on the collagen activator provided by the disclosure, and result shows that when a content of the collagen activator is 0.1%-1%, the collagen activator promotes an expression amount of the collagen I, and the expression amount of the collagen I reaches the highest when the content of the collagen activator is 0.5%; when a content of the collagen activator is 0.1%-0.5%, the collagen activator promotes an expression amount of the collagen XVII, and the expression amount reaches the highest when the content of the collagen activator is 0.4%; and when a content of the collagen activator is 0.1%-0.5%, the collagen activator promotes an expression amount of the collagen III, and the expression amount reaches the highest when the content of the collagen activator is 0.5%. The disclosure also performs a research on a SOX2 gene, which is used to regulate anti-aging, and results show that the collagen activator has a certain promotion effect on an expression amount of the SOX2 gene. Furthermore, when a content of the collagen activator is 0.5%, the expression amount of the SOX2 gene reaches the highest. In addition, cell proliferation and scratch experiments show that when the content of the collagen activator is 0.1%-1%, the collagen activator has an obvious promoting effect on the cell proliferation and cell migration.

Compared with the related art, the disclosure has the following beneficial effects.

According to the disclosure, the dipotassium glycyrrhizinate and the sodium acetylated hyaluronate are used as two main functional raw materials of the collagen activator, and the semen coiois extract, the Bletilla striata extract, and the astragalus extract are added according to the certain amount at the same time, thereby making the collagen activator provided by the disclosure capable of promoting the expressions of the endogenous collagen I, the endogenous collagen III, and the endogenous collagen XVII in the skin. Furthermore, the disclosure can regulate the anti-aging gene SOX2 to promote the cell proliferation and the cell migration, thereby achieving the effects of anti-aging and skin repairing.

The collagen activator provided by the disclosure not only can promote the amount of the endogenous collagens in the skin, but also has few types of raw materials, which leads to a low cost and a relatively high safety. In addition, the disclosure is used in a smearing mode, which is convenient and suitable for popularization and use.

### BRIEF DESCRIPTION OF DRAWINGS

In order to more clearly illustrate embodiments of the disclosure or technical solutions in the related art, attached drawings that need to be used in the embodiments are briefly described below. Apparently, the attached drawings in the following description are merely some embodiments of the disclosure, and those skilled in the related art can obtain other drawings according to the attached drawings without involving any inventive effort.

FIG. 1A illustrates an effect diagram of a skin efficacy test on a right face of a No. 8 volunteer before using a collagen activator prepared in an embodiment 1.

FIG. 1B illustrates an effect diagram of the skin efficacy test on the right face of the No. 8 volunteer after using the collagen activator prepared in the embodiment 1 for 4 weeks.

FIG. 1C illustrates an effect diagram of the skin efficacy test on the right face of the No. 8 volunteer after using the collagen activator prepared in the embodiment 1 for 8 weeks.

FIG. 1D illustrates an effect diagram of the skin efficacy test on the right face of the No. 8 volunteer after using the collagen activator prepared in the embodiment 1 for 9 weeks.

FIG. 2A illustrates an acquisition result diagram in a skin efficacy test of a No. 9 volunteer before using the collagen activator prepared in the embodiment 1.

FIG. 2B illustrates an acquisition result diagram in the skin efficacy test of the No. 9 volunteer after using the collagen activator prepared in the embodiment 1 for 4 weeks.

FIG. 2C illustrates an acquisition result diagram in the skin efficacy test of the No. 9 volunteer after using the collagen activator prepared in the embodiment 1 for 8 weeks.

FIG. 2D illustrates an acquisition result diagram in the skin efficacy test of the No. 9 volunteer after using the collagen activator prepared in the embodiment 1 for 9 weeks.

FIG. 3 illustrates a result diagram by an authority of a skin efficacy test of a No. 003 subject by using the collagen activator prepared in the embodiment 1.

FIG. 4 illustrates a result diagram by an authority of a skin efficacy test of a No. 006 subject by using the collagen activator prepared in the embodiment 1.

FIG. 5 illustrates a result diagram by an authority of a skin efficacy test of a No. 013 subject by using the collagen activator prepared in the embodiment 1.

FIG. 6 illustrates a result diagram by an authority of a skin efficacy test of a No. 017 subject by using the collagen activator prepared in the embodiment 1.

### DETAILED DESCRIPTION OF EMBODIMENTS

Various exemplary embodiments of the disclosure are now described in detail, which are not to be considered as a limitation to the disclosure, but are to be understood as a more detailed description of certain aspects, characteristics, and embodiments of the invention. It should be understood that the terminology described in the present invention is for the purpose of describing particular embodiments only and is not intended to be limiting of the invention.

In addition, for numerical ranges disclosed in the disclosure, it should be understood that each intermediate value between upper and lower limits of each of the numerical ranges is also specifically disclosed. Medium values within a stated value or a stated range and any other stated values or each smaller range between the medium values within the range are also included within the disclosure. The upper and lower limits of these smaller ranges may be independently included or excluded.

Unless otherwise stated, all technical and scientific terms used herein have the same meaning as commonly understood by those skilled in the related art of the disclosure. While the disclosure only describes specific methods and raw materials, any methods and raw materials similar or equivalent to those described herein can also be used in practice or testing of the disclosure. All of literature mentioned in the specification is incorporated by reference to disclose and describe methods and/or raw materials related to the literature. When there are conflicts between any incorporated literature and the disclosure, contents of the specification disclosed in the disclosure shall prevail.

Various modifications and variations can be made to the specific embodiments of the description of the disclosure without departing from the scope or spirit of the disclosure, which is apparent to those skilled in the related art. Other embodiments obtained from the specification of the disclosure will be apparent to those skilled in the art. The specification and embodiments of the disclosure are exemplary only.

As used herein, “comprising”, “including”, “having”, “containing”, etc. are all open terms, i.e., meaning including but not limited to.

In the following embodiments, semen coiois extract, Bletilla striata extract, and astragalus extract are all purchased from Xi'an Shouhe Biotechnology Co., Ltd, all of which are solid extracts. The description of the extracts will not be repeated below.

### Embodiment 1

A collagen activator is prepared by using the following raw materials in percentage by mass:

15% of butylene glycol (C₄H₁₀O₂), 2% of 1, 2-hexanediol (C₆H₁₄O₂), 1% of dipotassium glycyrrhizinate (C₄₂H₆₃KO₁₆), 1% of sodium acetylated hyaluronate, 0.1% of semen coiois extract, 0.01% of Bletilla striata extract, 0.01% of astragalus extract, and 80.88% of water.

A preparation method for the collagen activator includes the following steps: weighing the above mentioned raw materials in percentage by mass, and then mixing and uniformly stirring the raw materials to obtain the collagen activator.

### Embodiment 2

A collagen activator is prepared by using the following raw materials in percentage by mass:

16% of butylene glycol, 3% of 1, 2-hexanediol, 1% of dipotassium glycyrrhizinate, 0.5% of sodium acetylated hyaluronate, 0.01% of semen coiois extract, 0.07% of Bletilla striata extract, 0.005% of astragalus extract, and 79.415% of water.

A preparation method for the collagen activator includes the following steps: weighing the above mentioned raw materials in percentage by mass, and then mixing and uniformly stirring the raw materials to obtain the collagen activator.

### Embodiment 3

A collagen activator is prepared by using the following raw materials in percentage by mass:

20% of butylene glycol, 1% of 1, 2-hexanediol, 2% of dipotassium glycyrrhizinate, 2% of sodium acetylated hyaluronate, 0.05% of semen coiois extract, 0.1% of bletilla striata extract, 0.007% of astragalus extract, and 74.843% of water.

A preparation method for the collagen activator includes the following steps: weighing the above mentioned raw materials in percentage by mass, and then mixing and uniformly stirring the raw materials to obtain the collagen activator.

### Comparative Embodiment 1

Compared with the embodiment 1, a difference between the comparative embodiment 1 and the embodiment 1 lies in replacing the dipotassium glycyrrhizinate with allantoin (C₄H₆N₄O₃).

### Comparative Embodiment 2

Compared with the embodiment 1, a difference between the comparative embodiment 2 and the embodiment 1 lies in replacing the sodium acetylated hyaluronate with sodium hyaluronate (C₁₄H₂₂NNaO₁₁).

### Effect Verification

### 1. Safety Test

Saline is added to the collagen activator prepared in the embodiment 1 to prepare collagen activator products with concentrations of 25 wt %, 35 wt %, 70 wt %, 50 wt %, and 100 wt %, respectively. And then, the collagen activator products are used to stimulate blood vessels of embryonated eggs (also referred to as chicken ataxias) in 9 days old, and 10 embryonated eggs are selected for adding the collagen activator product with each concentration. It is observed that whether there is a bleeding phenomenon in the blood vessels in the embryonated eggs or not, thereby evaluating safety performances of the collagen activator products with different concentrations.

A negative blank comparative group includes: using saline to stimulate 5 embryonated eggs.

A positive comparative group includes: using 0.05 g/mol (M) sodium dodecyl sulfate (C₁₂H₂₅NaO₄S, abbreviated as SDS) to stimulate 5 embryonated eggs.

A specific implementation scheme for the collagen activator products, the negative blank comparative group, and the positive comparative group is as follows: after an embryonated egg is incubated for 9 days, selecting the embryonated egg with well-developed blood vessels by using an egg light, making a small hole in an air cell of the embryonated egg, peeling off a part of a shell of the embryonated egg located at the air cell, transferring the embryonated egg to an ultra-clean workbench, adding a small amount of saline to a film of the air cell of the embryonated egg, absorbing the saline after the film is completely wetted, removing the film with tweezers, placing a Teflon® ring on the blood vessels of the well-developed embryonated egg, and then dropping 40 microliters (L) of a corresponding test solution (i.e., the collagen activator products with different concentrations, the saline used in the negative blank comparative group, and the 0.05 M SDS used in the positive comparative group) to react for 30 minutes (min).

Results are shown as follows. The blood vessels of the 5 embryonated eggs in the negative blank comparative group appear no bleeding phenomenon, indicating that there are non-irritants. The blood vessels of the 5 embryonated eggs in the positive comparative group (adding with 0.05 M SDS) appear the bleeding phenomenon, indicating that there are non-irritants. Furthermore, in the group of the 10 embryonated eggs using the collagen activator with the concentration of 100 wt %, 3 embryonated eggs are normal, 1 embryonated egg has a moderate hemorrhaging phenomena, i.e., small area bleeding coverage 50%, and 6 embryonated eggs have a slight hemorrhaging phenomena, i.e., small bleeding points covering an area of 25%-50%; according to a judgment standard RC₅₀, the collagen activator with the concentration of 100 wt % has non-irritants. It is noted that the judgment standard RC₅₀ refers to when 50% of the embryonated eggs show positivity (i.e., the bleeding phenomenon) and a final concentration of the test solution is greater than 3%, the test solution has non-irritants; otherwise, the test solution has irritants. In the group of the 10 embryonated eggs using the collagen activator with the concentration of 70 wt %, 7 embryonated eggs are normal, 1 embryonated egg has a capillary injection phenomenon, and 2 embryonated eggs have a slight hemorrhaging phenomena, i.e., small bleeding points covering an area of 25%-50%, indicating that there are non-irritants. In the group of the 10 embryonated eggs using the collagen activator with the concentration of 50 wt %, 7 embryonated eggs are normal, 1 embryonated egg has a minimal hemorrhaging phenomenon, 1 embryonated egg has a slight hemorrhaging phenomenon, 1 embryonated egg has ghost vessels, and the judgment standard RC₅₀ is greater than 3%, indicating that there are non-irritants. In the group of the 10 embryonated eggs using the collagen activator with the concentration of 35 wt %, 1 embryonated egg has ghost vessels, the remaining 9 embryonated eggs are normal, and the judgment standard RC50 is greater than 3%, indicating that there are non-irritants. In the group of the 10 embryonated eggs using the collagen activator with the concentration of 25 wt %, 10 embryonated eggs are all normal, and therefore, there are non-irritants. The above results illustrate that the formulation of the disclosure has certain safety. Furthermore, a further verification is performed on collagen activator products with concentrations of less than 25 wt % according to the above implementation scheme, and embryonated eggs are normal, indicating that the products have non-irritants.

Along with richness and modernization of detection and evaluation means, experimental materials of an embryonated egg experiment are easy to obtain, an operation of the embryonated egg experiment is simple, an experimental period is short, and no special equipment is needed. Meanwhile, a price of the embryonated egg experiment is much lower than that of a mouse experiment using an ordinary strain, thereby greatly reducing the experiment cost.

### 2. Skin Efficacy Test

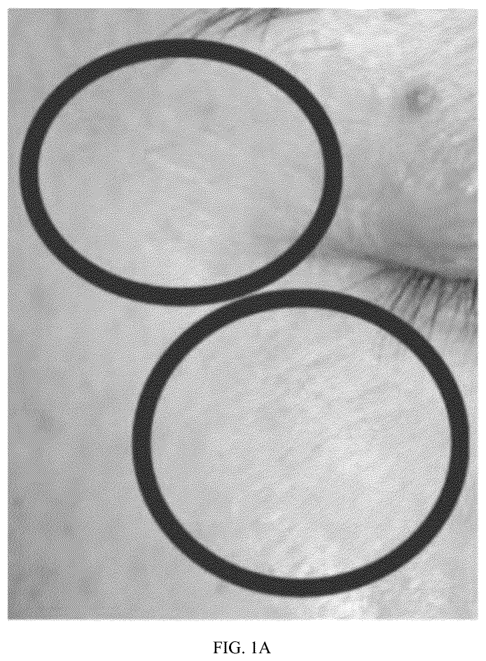

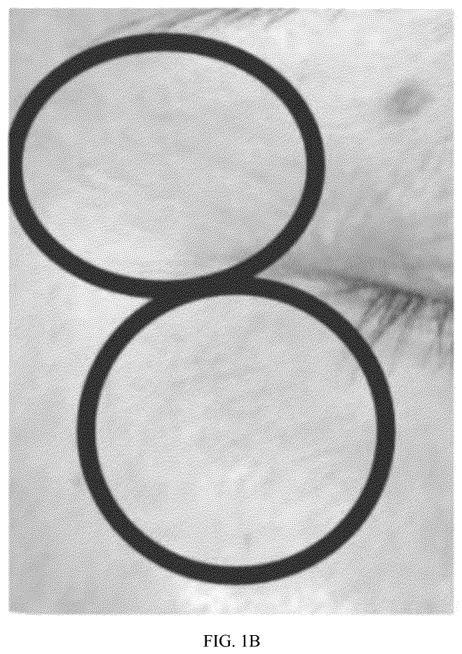

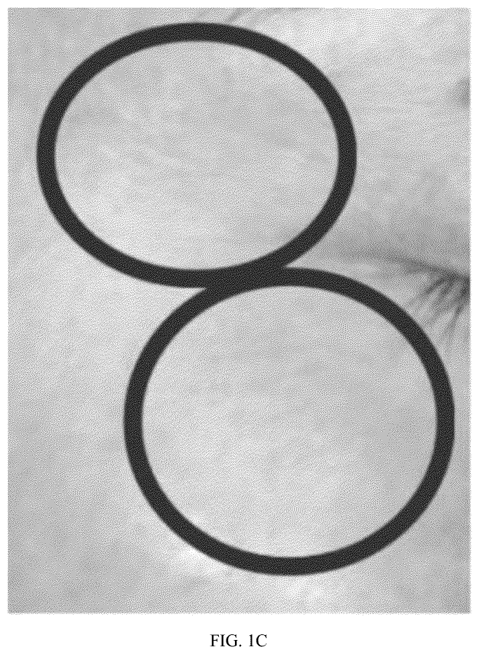

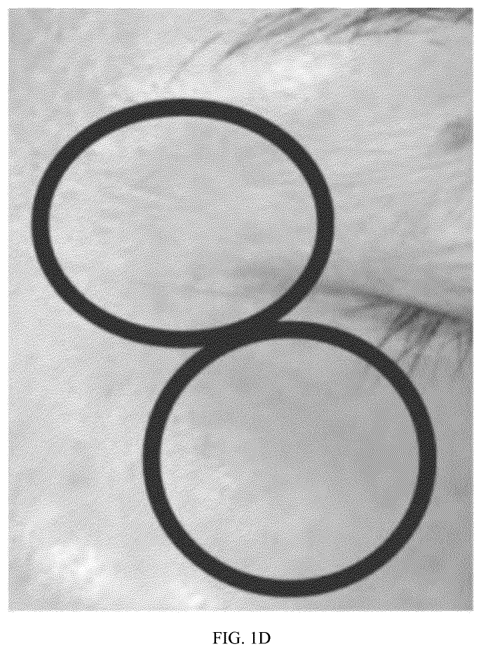

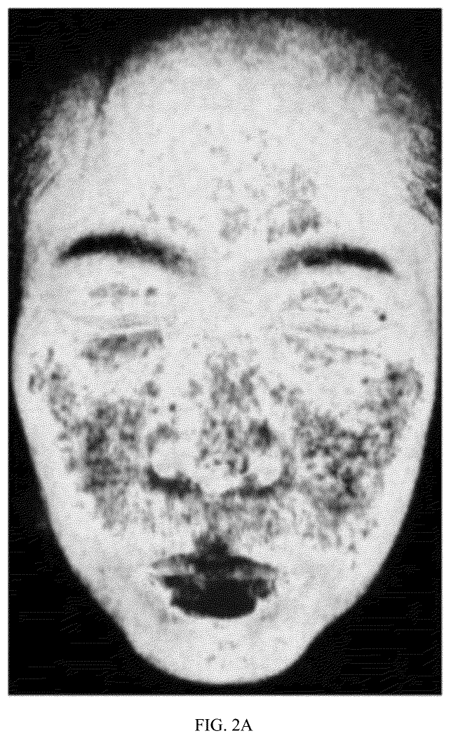

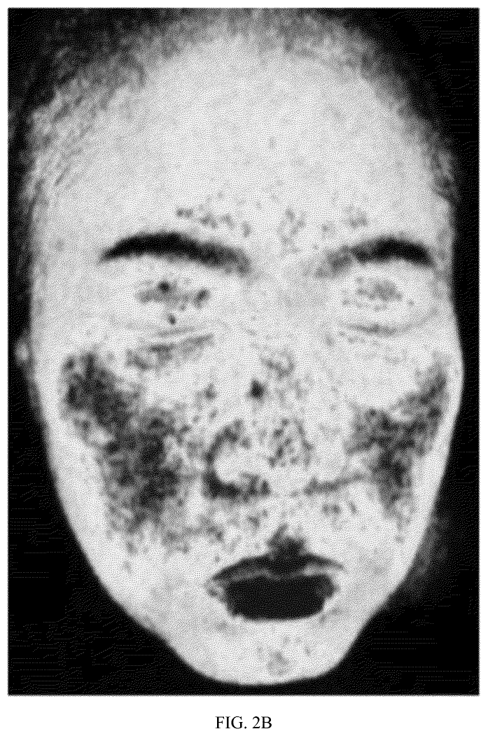

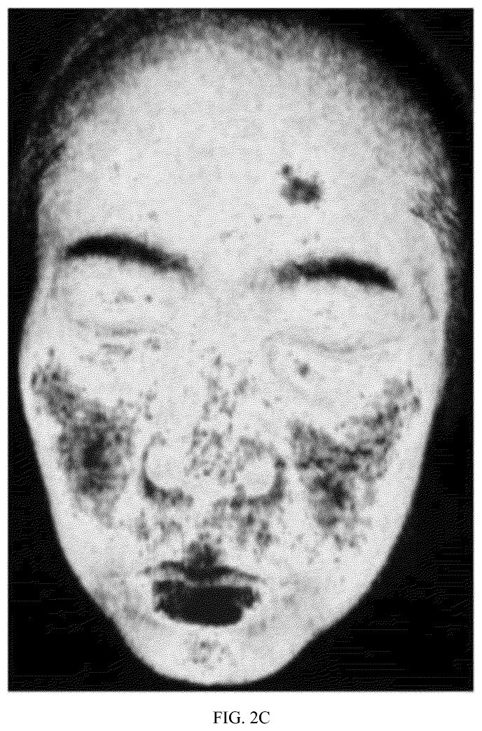

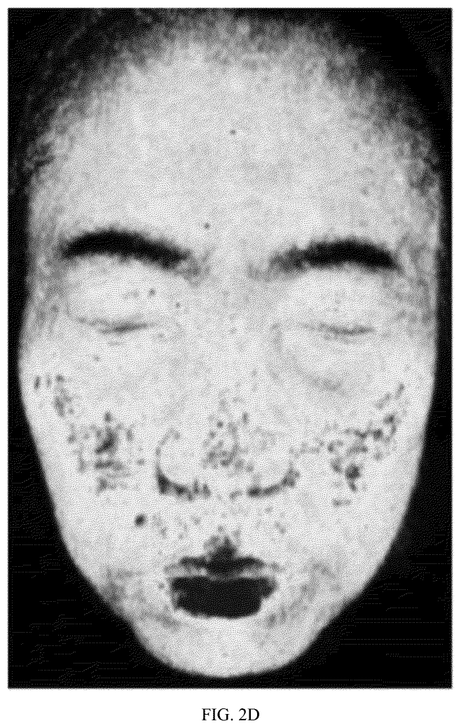

The collagen activator prepared in the embodiment 1 is added as a base material of a skincare product to prepare a test article with content of the collagen activator of 10 wt %. 10 female volunteers at 40 years old are selected and numbered 1-10, and the test product is tested for up to 9 weeks. Each skin test is performed after skin cleaning in warm water (at 37 degrees Celsius abbreviated as ° C. to 42° C.). Images of front faces, left faces, and right faces of the subjects are collected by using a beauty testing skin detection system MC-880 to analyze wrinkles. The subjects' skin are tested and compared before and after 0 week, 2 weeks, 4 weeks, 8 weeks, and 9 weeks. After reviewing the results, it is found that after 2 weeks of smearing, the face skin state of the subject is not obviously changed; after 4 weeks of smearing, the face skin state of the subject is not obviously changed; after 8 and 9 weeks of smearing, the skin wrinkle severity of the face of the subject is obviously improved. Furthermore, the effect diagrams of the No. 8 volunteer before use, after 4 weeks of use, after 8 weeks of use, and after 9 weeks of use on the right face of the above test item are shown in FIGS. 1A-1D. Front image acquisition results of the No. 8 volunteer are shown in FIGS. 2A-2D, where FIGS. 2A-2D are image acquisition results before using the test article, after using for four weeks, after using for 8 weeks, and after using for 9 weeks.

Another 10 40-year-old female volunteers are selected, and the facial wrinkle conditions thereof are substantially equivalent to those of the No. 8 volunteer. The products obtained from the comparative embodiments 1-2 are subjected to the skin efficacy test by using the same above mentioned method, and it is found that the products prepared in the comparative embodiments 1-2 do not have an obvious anti-wrinkle effect.

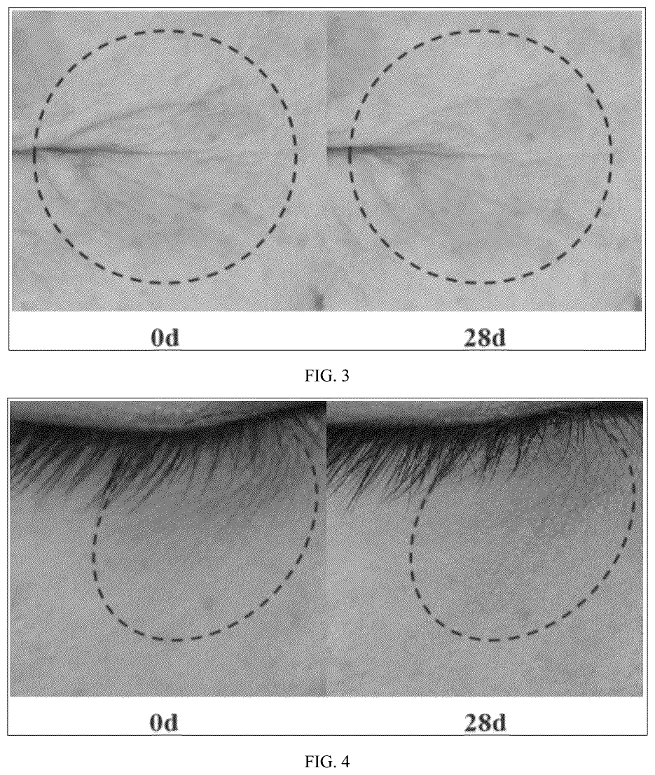

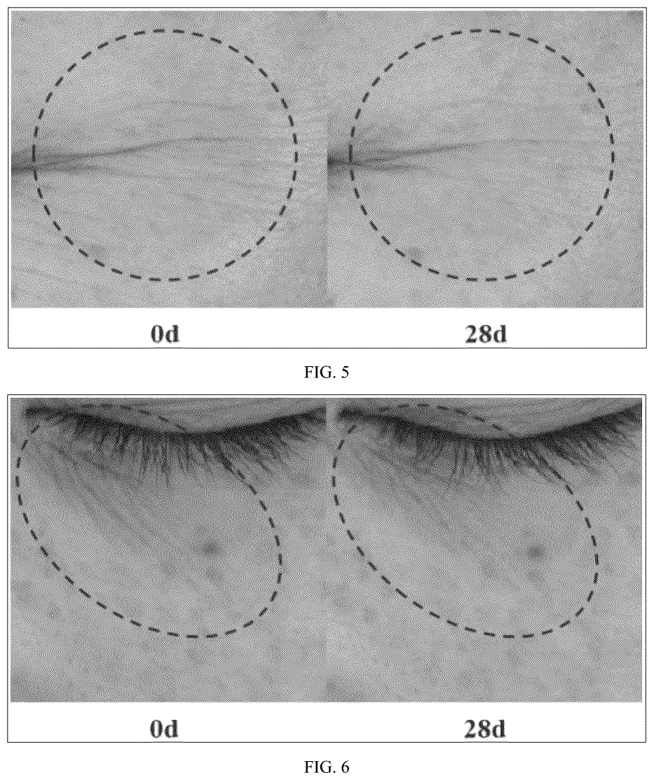

The collagen activator prepared in the embodiment 1 is subjected to an authority for the detection. 28 healthy subjects aged at 30-65 years old, whose skin is lax and lack of elasticity and crow's feet is at 2-4 level are numbered as 001-028, and then a side of the face of the subject is randomly selected as an experimental side, and the other side is used as a blank control. After the subjects clean their faces in the morning and evening every day, the experimental side uses the test article (i.e., adding the collagen activator prepared in the embodiment 1 to a blank substrate to prepare a test prepare the test article with the collagen activator content of 10 wt %), while the control side uses a blank substrate. During the entire testing period, the subjects are not allowed to engage in prolonged exposure to sunlight, outdoor activities, travel, etc., and are not allowed to use cosmetics or drugs with similar efficacy to the test article. During the testing period, the subjects are not allowed to change their daily skincare habits. After 28 days, a comparison is made between the experimental side and the control side of the subject's face, and a comparison is also made on the subject's face before and after using the test article. And then, the comparison results show that on the experimental side, a wrinkle area around the eye is significantly reduced by 7.52% (p<0.001, i.e., a significant difference) after using the test article, compared with before using the test article. However, on the control side, there is no significant decrease in the eye wrinkle area between before using the blank substrate and after using the blank substrate for 28 days. Furthermore, comparing the experimental side with the control side, the difference on the experimental side is significantly lower than that on the control side (p<0.001). On the experimental side, comparing before using the test article with using the test article for 28 days, a wrinkle area below the eye is significantly reduced by 16.42% (p<0.001). However, on the control side, there is no significant decrease in the eye wrinkle area between before using the blank substrate and after using the blank substrate for 28 days. Furthermore, comparing the experimental side with the control side, the difference on the experimental side is significantly lower than that on the control side (p<0.001). FIGS. 3-6 illustrate the effect comparison diagrams of the facial experimental sides of the subjects numbered 003, 006, 013, and 017 before and after the test, respectively. As shown in FIGS. 3-6, after 28 days of the experiment, the facial wrinkle area around and below the eyes of the subjects' experimental side significantly decreases.

The collagen activators prepared in the embodiments 2-3 perform the safety and skin efficacy tests according to the above-mentioned method and the obtained results are basically consistent with those of the collagen activator prepared in the embodiment 1.

The collagen activator provided by the disclosure can improve severe skin aging, has good permeability, and is safe and non-irritant in use. The formula of the disclosure includes the dipotassium glycyrrhizinate capable of promoting the absorption of nutrients by the skin, especially minerals and trace elements, promoting skin metabolism, removing metabolic waste such as oxygen radicals, making the skin elastic and glossy, and having the effects of removing wrinkles. Another main functional raw material is the sodium acetylated hyaluronate, which has moisturizing and anti-aging effects, repairs skin injury, promotes proliferation and differentiation of skin cells, removes oxygen free radicals, delays skin aging, and also has the effects of crosslinking, supporting and filling to repair the barrier on the skin surface, and promotes the synthesis of collagen, thereby effectively promote skin recovery. The anti-aging cosmetic for reducing wrinkling rate, i.e., the disclosure, comprehensively utilizes the above described two raw materials, enabling the cosmetic to effectively repair cells, to moisten and moisturize, to fade faint lines, to reduce wrinkles, to play a role in anti-aging, and to increase skin elasticity, leading to the skin to glow. In addition, among the added Chinese herbal medicine mixture, the semen coiois extract can whiten the skin and resist oxidation in a moderate degree, can also increase skin activity, reduce wrinkles, and have an anti-aging effect; the Bletilla striata extract can prevent skin from cracking, promote blood circulation, and improve skin nutrition; and the astragalus extract can expand skin blood circulation, delay skin aging, and supplement necessary trace elements of the human body.

The above only describes the specific embodiments of the disclosure, and the scope of the protection of the disclosure is not limited thereto. Those skilled in the related art familiar with the technical field can make equivalent replacements or changes to the disclosure within the scope disclosed by the disclosure based on the technical solutions of the disclosure and the inventive concept thereof. Therefore, these replacements or changes shall be covered within the scope of the protection of the disclosure.

What is claimed is:

1. A collagen activator, consisting of the following raw materials by mass percentage: 15%-20% of butylene glycol (C₄H₁₀O₂), 1%-4% of 1, 2-hexanediol (C₆H₁₄O₂), 0.5%-2% of dipotassium glycyrrhizinate (C₄₂H₆₃KO₁₆), 0.1%-2% of sodium acetylated hyaluronate, 0.01%-0.1% of semen coiois extract, 0.01%-0.1% of Bletilla striata extract, 0.001%-0.01% of astragalus extract, and the balance of water.

2. A preparation method of the collagen activator as claimed in claim 1, comprising the following steps: weighing the raw materials and mixing the raw materials to obtain the collagen activator.

3. An application method of the collagen activator as claimed in claim 1 in a skincare product.

4. The application method as claimed in claim 3, wherein the skincare product is an anti-aging skincare product.

5. An anti-aging skincare product, comprising the collagen activator as claimed in claim 1.

6. The anti-aging skincare product as claimed in claim 5, wherein a content of the collagen activator is in a range of 3 wt % to 15 wt %.
